(19) United States
(12) Patent Application Publication
(10) Pub. No.: US 2022/0000184 A1
(43) Pub. Date: Jan. 6, 2022

(54) VAPE TOOL AND VAPE TOOL KIT FOR MAKING VAPING COILS

(71) Applicant: Graph-X Limited, Halesowen Birmingham (GB)

(72) Inventors: Christopher Sheppard, Halesowen Birmingham (GB); Benjamin Drinkwater, Halesowen Birmingham (GB)

(21) Appl. No.: 17/291,506

(22) PCT Filed: Nov. 15, 2019
(86) PCT No.: PCT/GB19/53240

§ 371 Date: May 5, 2021

(30) Foreign Application Priority Data
Nov. 15, 2018 (GB) 1818614.8

(51) Int. Cl. A24F 40/70; B21F 3/04; A24F 40/46

(52) U.S. Cl. CPC A24F 40/70; A24F 40/46; B21F 3/04

(57) ABSTRACT

A vape tool comprises a coiling rod 114, and a coiling tool 94 for engaging with and rotating around the coiling rod 114, the coiling tool 94 having a housing 96 with a substantially conical inner wall, a collet 106 disposed inside the housing 96 and arranged to bear against the inner wall, an internal diameter of the collet 106 being determined by the longitudinal position of the collet within the housing and a control means 112 for controlling the longitudinal position of the collet 106 within the housing 96. The tool 94 forms part of a vape tool kit (10, FIG. 1) including a four part housing for housing a tool barrel (22, FIG. 1), a spool (44, FIG. 1) and a sealable container.

The present invention relates to a vape tool and vape tool kit for making vaping coils.

### BACKGROUND TO THE INVENTION

Vaping is a highly popular recreational activity, which is being favoured over smoking. It is estimated by the World Health Organisation that the number of people vaping by the end of 2016 had grown to around 35 million people, from 7 million in 2011. It is known to provide tool kits for making vaping coils for vaping, and making coils is regarded by many as a fun and creative part of the vaping experience.

A problem with current vape tool kits is that they are quite large and tend to be provided in soft shell cases, with zip fastener extending around the periphery of the casing. The tool kits take the appearance of old-fashioned soft-shell pencil cases and tools, such as pliers, tweezers, cutters, screwdrivers and coiling rods are held in place by elastic.

Specific problems arise in the storing of cotton, which must be kept dry and clean. Furthermore, the vaping wire can easily become unravelled and damaged, since it is supplied on open reels. It is also often wound by hand around a coiling rod or the stem of a screwdriver. More sophisticated kits include coiling rods with apertures for securing an end of the wire whilst the wire is rotated around the rod, and some include wire guides for guiding the wire being coiled.

It is an object of the present invention to provide a vape tool and vape tool kit which reduces or substantially obviates the aforementioned problems.

### STATEMENT OF INVENTION

According to the present invention there is provided a vape tool comprising a coiling rod, and a coiling tool for engaging with and rotating around the coiling rod, the coiling tool having a housing with a substantially conical inner wall, a collet disposed inside the housing and arranged to bear against the inner wall, an internal diameter of the collet being determined by the longitudinal position of the collet within the housing and a control means for controlling the longitudinal position of the collet within the housing.

The tool provides a simple and convenient way of forming coils of the same size and quality. Furthermore, the collet arrangement can accept different diameters of coiling rod.

The control means may be a thumb screw threadingly engaged with the housing and connected to the collet. An aperture may be provided in an end of the housing for receiving the coiling rod.

A coiling rod holder may be provided for use with the coiling rod and coiling tool. The coiling rod holder may have a body which is separate to or discrete from the other components of the vape tool. The coiling rod holder may be of similar size to the coiling tool. This allows the coiling assembly (tool, rod and rod holder) to be more easily held or manually manipulated when making a coil. The coiling rod holder may be a cylinder or may be substantially cylindrical for ease of rotation during use.

A coiling rod aperture may be provided in an end of the coiling rod holder for receiving the coiling rod. The coiling rod aperture may include a substantially hexagonal or non-circular portion for receiving a substantially hexagonal or non-circular end of the coiling rod.

A first hole for guiding coiling wire may be provided adjacent to the coiling rod aperture. A first corresponding hole may be provided on a side or another end of the coiling rod holder. The two holes may be connected together (e.g. via a bore) through the body of the coiling rod holder. The bore should be at least as wide as the diameter of the coiling wire.

A second hole may be provided adjacent to the coiling rod aperture. The second hole may be on the opposite side of the coiling rod aperture to the first hole. A second corresponding hole may be provided on a side or another end of the coiling rod holder. The two second holes may be connected together (e.g. via a bore) through the body of the coiling rod holder. This means that the coiling rod holder is suitable for use in either hand.

At least one slot or notch or cut-out may be provided across the top of the coiling rod holder. The slot may be offset to one side of the coiling rod aperture. The slot may extend into the holder on a plane or axis which is substantially parallel to the axis of the coiling rod aperture. In some embodiments, two slots may be provided on opposite sides of the top of the coiling rod holder for left or right-handed use. The slot(s) can receive and guide flat ribbon (or ribbon-type wire) when making a coil from the ribbon.

The coiling rod holder may be magnetic or magnetised. A magnet or magnets may disposed in the coiling rod holder. The magnet(s) may be disposed adjacent to the coiling rod aperture. The magnet(s) may be disposed at or adjacent to the base of the coiling rod aperture. Where the coiling rod is magnetic, or made of (or includes) a magnetic or magnetisable material, the coiling rod can be held more securely in the coiling rod holder. Where a coil is tightened too much, having the coiling rod secured in the holder by a magnet means that the rod can be more easily withdrawn from the coil, instead of the rod being clamped within the coil and trapped in situ within the coil.

A wire guide may be disposed at an end of the housing for guiding wire around the coiling rod on rotation of the coiling tool relative to the coiling rod. The wire guide may be a screw.

The vape tool may be part of a kit including a casing. The casing may include a central body portion, an upper end cap and a lower end cap. The upper end cap may be securable to one end of the central body portion. The lower end cap may be securable to the other end of the central body portion.

One or both of the upper and lower end caps may include knurling or longitudinal slots on an outer side of that end cap, for improving grip when screwing or unscrewing the end cap.

A spool for storing vaping wire may be disposed within the central body portion and/or lower end cap.

The vape tool may be part of a kit including a casing, the casing having a central body portion, an upper end cap securable to one end of the central body portion, an intermediate cap securable to the other end of the central body portion and a lower end cap securable to the intermediate cap. Conveniently the casing may be cylindrical and sized to fit in a pocket.

An aperture may be provided in the intermediate cap for receiving a coiling rod. The end of the coiling rod received in the intermediate cap may be hexagonal and the aperture in the intermediate cap may be correspondingly hexagonal.

Advantageously one end of the coiling rod can be located in the intermediate cap, whilst the other end is located in the coiling tool. The hexagonal engagement serves to prevent rotation of the rod relative to the cap. It will be appreciated that other non-circular shapes may be provided for the end of the coiling rod and rod holder or portion of the intermediate cap. A tool barrel for storing tools may be disposed within the central body of the casing.

A spool for storing vaping wire may be disposed within the central body portion and intermediate cap. The spool may have external protrusions spaced around the spool for retaining a winding of vaping wire.

A sealable area for containing cotton may be formed within the lower end cap. The sealable area may be partitioned to provide a space for cotton and a space for screws and pre-made vaping coils.

According to a second aspect of the invention there is provided a vape tool kit comprising a casing including a central body portion and an upper end cap attachable to one end of the central body portion and enclosing a first space, an intermediate cap enclosing a second space, and a lower end cap enclosing a third space, a tool kit being located in the first space, a spool and sealable cotton storage area being located in the second and third spaces, in which the intermediate cap is attachable to the other end of the central body portion and the lower end cap is attachable to the intermediate cap, or in which the intermediate cap is disposable in the lower end cap and the lower end cap is attachable to the central body portion.

Advantageously the vape tool kit is modular, in that a number of cylindrical elements are connected together. It is envisaged that the tool kit could be extended, for example, by adding one or more further modules. For example, a further intermediate module could be positioned between the central body portion and the upper end cap.

The tool kit may include a cylindrical barrel, having apertures formed therein for receiving tools. The tool barrel may be made from silicone rubber. The tool barrel may be disposed within the first space.

The spool may be formed from upper and lower parts and may include a U-shaped circular wall onto which wire can be coiled. The spool may have a cover element which may lie over the U-shaped circular wall for protecting coiled wire on the spool.

External protrusions may be provided on the spool for temporarily receiving a second coil of wire.

A cover or cover plate may close a space in the lower end cap and may form the sealable cotton storage area. The cover or cover plate may be provided with a peripheral seal.

The upper end cap may be screwed to one end of the central body portion. The intermediate cap may be screwed to the other end of the central body portion. The lower end cap may be screwed to the intermediate cap. In some instances, the lower end cap may be screwed to another end of the central body portion. The intermediate cap may be disposed inside the lower end cap.

According to a third aspect of the invention there is provided a vape tool, kit comprising a casing including a central body portion and an upper end cap attachable to one end of the central body portion and enclosing a first space, an intermediate cap attachable to the other end of the central body portion and enclosing a second space, and a lower end cap attachable to the intermediate cap and enclosing a third space, a tool kit being located in the first space, a spool and sealable cotton storage area being located in the second and third spaces.

Any feature or combination of features presented with respect to the first, second or third aspects of the invention may be included in any of the other aspects of the invention.

### BRIEF DESCRIPTION OF THE DRAWINGS

For a better understanding of the present invention, and to show more clearly how it may be carried into effect, reference will now be made by way of example only to the accompanying drawings, in which:

FIG. 1 shows a cross sectional view of a tool for making vaping coils;

FIG. 2 shows an upper end of a tool cartridge forming part of the tool of FIG. 1;

FIG. 3 shows the lower end of the tool cartridge of FIG. 2;

FIG. 4A shows a spool forming part of the tool of FIG. 1;

FIG. 4B shows the spool of FIG. 1 in a disassembled condition for loading with wire;

FIG. 5 shows a lower end cap of the tool of FIG. 1;

FIG. 6 shows a cross sectional side view of the manufacture of a vaping coil using a collet clamp and coiling rod of the tool of FIG. 1;

FIG. 7 shows a part perspective, part cross sectional view of the collet clamp and coiling rod of FIG. 6;

FIG. 8 shows a perspective view of a coiling rod holder;

FIG. 9 shows a cross-sectional side view of the coiling rod holder of FIG. 8 engaged with a coiling rod and coiling tool;

FIG. 10 shows a perspective view of the top side of a cover for the lower end cap; and

FIG. 11 shows another perspective view of the cover of FIG. 11.

### DESCRIPTION OF PREFERRED EMBODIMENTS

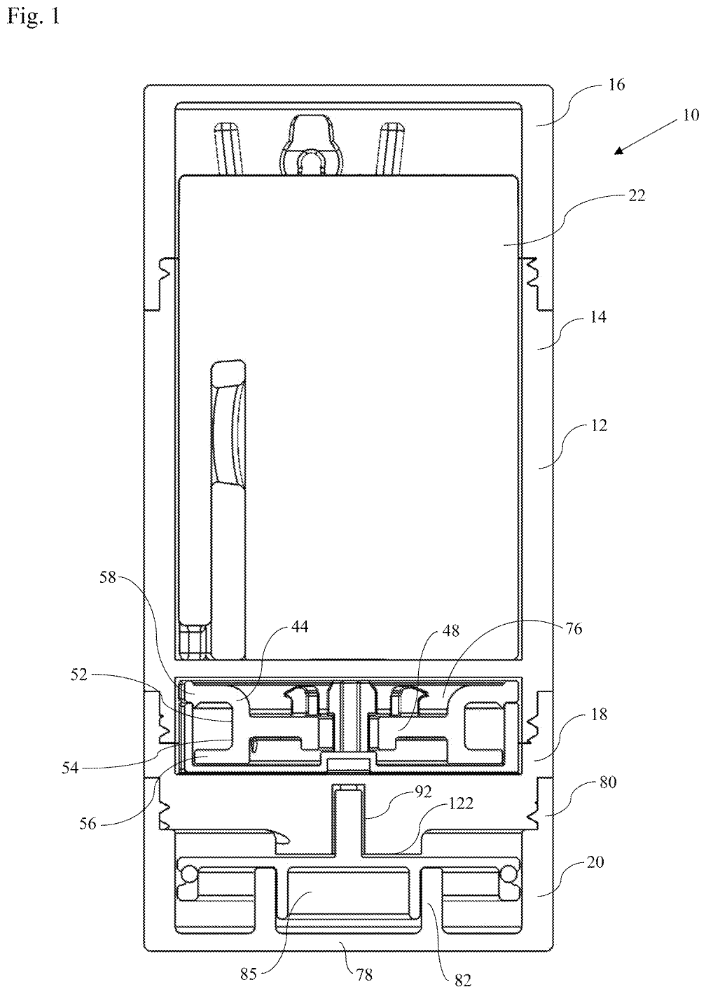

Referring firstly to FIG. 1, a vape tool kit is indicated generally at 10. The kit 10 is enclosed in a housing 12. The housing 12 is cylindrical and is designed to fit in a person's pocket. In one embodiment, it is 150 mm in length with a diameter of 55 mm. The housing 12 is made from metal, preferably anodised aluminium, and is waterproof, to protect the contents. A rigid plastic may also be utilised for the housing 12. In other words, it is completely sealed to prevent ingress of dirt and/or moisture.

The housing has a central body portion 14, an upper end cap 16, an intermediate cap 18 and a lower end cap 20. The central body portion 14 and upper end cap 16 define a cavity, which houses a tool holder or barrel 22, preferably made from a firm silicone. The tool barrel 22 fits snugly within the housing 12, but can be removed, when the upper end cap 16 is detached from the central body portion.

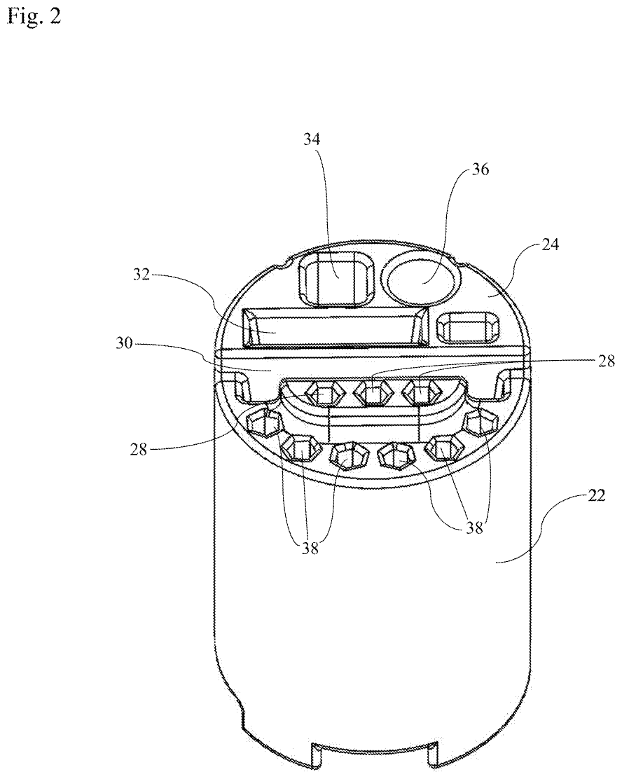

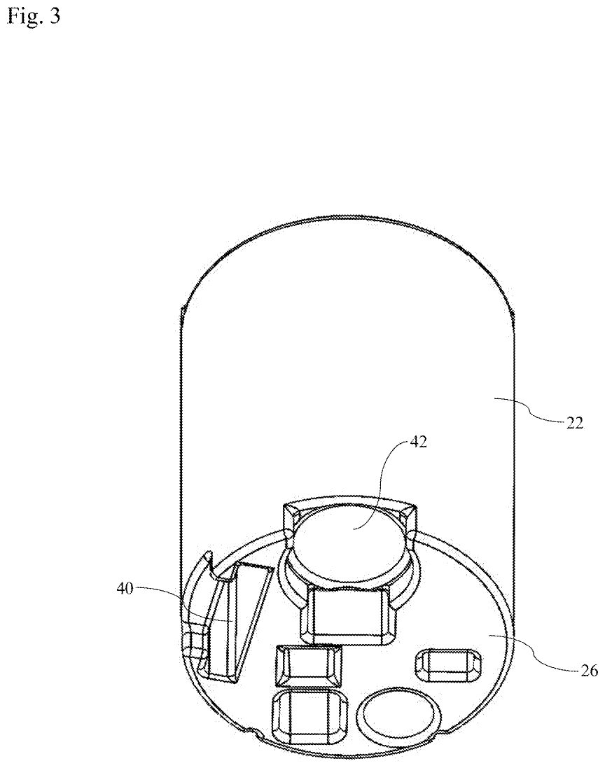

Referring also to FIGS. 2 and 3, the tool barrel 22 is provided with a number of tool apertures in the upper end 24 and the lower end 26. In the upper end 24, there are three apertures 28 for flat head, hexagon and cross head screwdriver bits, a slot 30 for a pliers, a slot 32 for wire cutters, an aperture 34 for ceramic or aluminium tweezers, an aperture 36 for a screwdriver, and a plurality of apertures 38 for different diameter coiling rods. A wire brush can also be located in one of the apertures 38, and if necessary, the shape of the aperture can be altered to have a substantially circular cross-section accordingly. A wire brush is typically used to clean the contacts of a vaping device.

Some of the apertures extend through the barrel 22, but in the lower end 26 of the tool barrel 22, there is an aperture 40 for receiving scissors, and an aperture 42 for a coiling tool. The scissors may be collapsible or foldable.

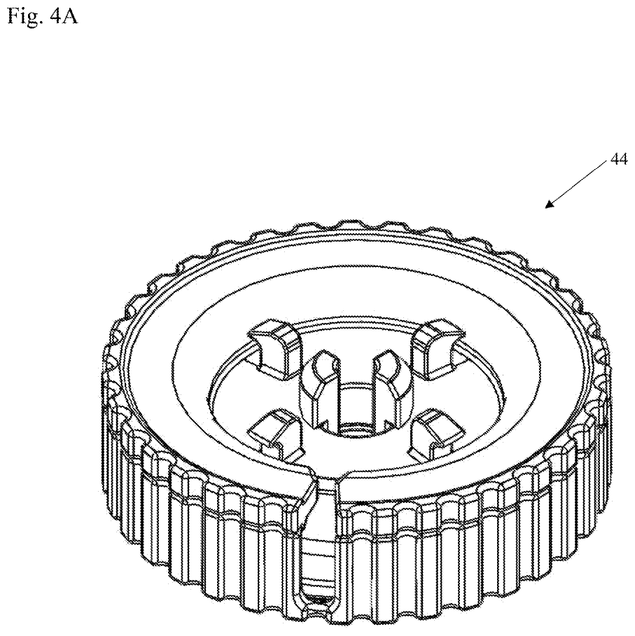

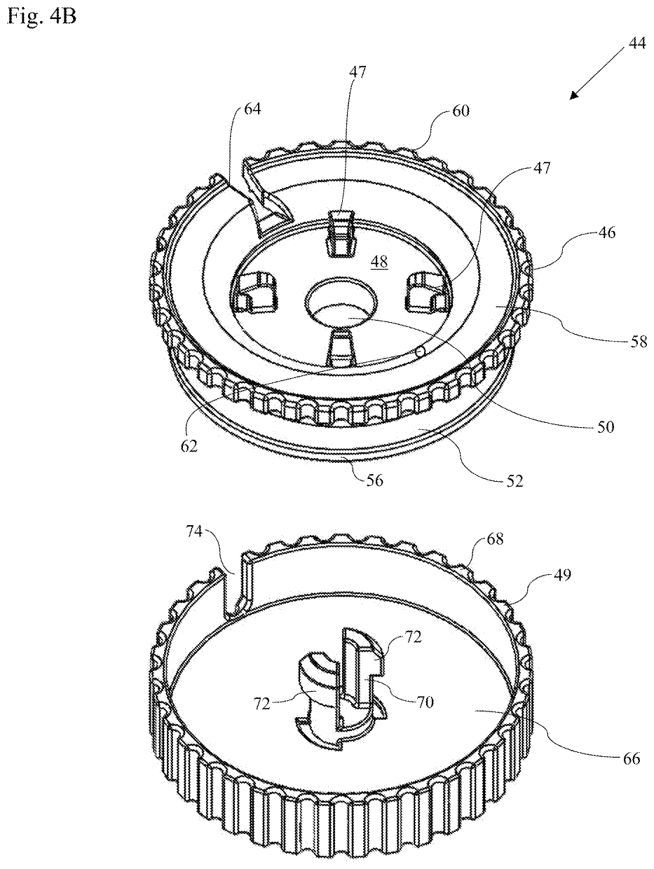

Referring to FIGS. 4A and 4B, a spool for vaping wire is indicated at 44. The spool 44 can also be seen in cross-section in FIG. 1. The spool 44 is formed from 2-parts, a spool element 46 and a cover element 48. The spool element 46 includes a central circular plate 48, with a circular aperture 50 at its centre. A circular wire holder 52 extends around the periphery of the circular plate and is substantially U-shaped. The U-shape includes an inner peripheral wall 54, a lower wall 56 and an upper wall 58. The upper wall 58 extends beyond the lower wall 56, and is provided with a grip portion 60 around its outer edge, formed by part circular recesses. An aperture 62 is provided through the upper wall 58 to a position adjacent the inner wall 54 for retaining the end of a wire to be wound on the coil.

Four hook-shaped protrusions 47 are equi-spaced around the circular plate 48, that is at 90 degrees apart. They terminate below the plane of the upper wall 58. The hooks face outwardly, and are positioned closer to the back of the inner peripheral wall 54 then the aperture 50. A slot 64 extends radially through part of the inner peripheral wall 54 and upper wall 58, disposed on one side of the circular plate 48.

The cover element 49 comprises a circular plate 66 with a circumferential flange 68. A resilient spigot 70 extends from the centre of the circular plate 66, to the same side as the flange 68. The resilient spigot 70 is not solid, but has a slot along part of its length to enable inward flexing. Protrusions 72 are provided at the end of the spigot 70, which are chamfered at their distal end to enable pushing through the aperture 50 of the spool element 46 and snap engagement against the circular plate 48, best seen in FIG. 4A.

A slot 74 is provided through the circumferential flange 68 and can be arranged to align with the slot 64, when the spool is assembled. The use of the spool 44 is described further below.

The spool 44 is located and stored in a space 76 located between the central body portion 14 and the intermediate cap 18. The space 76 is cylindrical and the spool 44 fits snugly within the space.

Referring now to FIGS. 1 and 5, the lower end cap 20 forms a cotton storage compartment. The lower end cap 20 has an end wall 78 adjoining a peripheral wall 80. Inside the end cap 20, a circular wall 82, concentric with the peripheral wall 80 extends upwardly for around half the height of the cap. This defines a space 84, in the form of a hoop between the peripheral wall 82 and inner circular wall 82, where cotton can be wound and stored. A circular cover plate 86 closes the space 84 and has a seal 88, preferably a nitrile seal, around its periphery. The seal prevents ingress of moisture and/or dirt and keeps the cotton clean. A space 85, inside the circular wall 82 is used to store screws and pre-formed coils. A handle 90, in the form of a cylindrical spigot, extends outwardly from the centre of the cover plate 86. When the lower end cap 20 is located on the intermediate cap 18, as shown in FIG. 1, the spigot 90 locates in a recess 92, disposed on the underside of the intermediate cap. The recess is hexagonal in cross-section, as described further below.

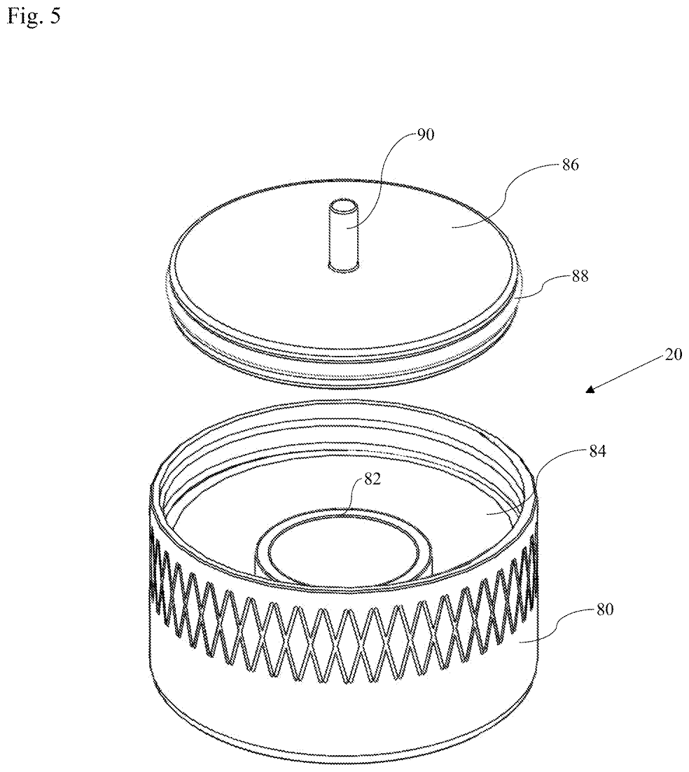

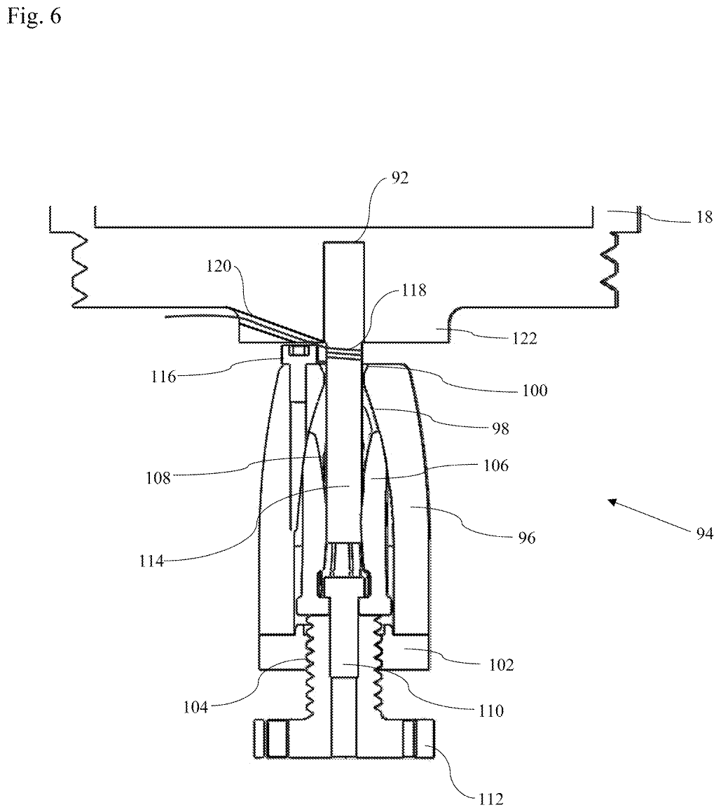

A coiling tool 94 will now be described with reference to FIG. 6. The coiling tool 94 is a clamping device utilising a collet design for holding coiling rods of different diameters. The coiling tool 94 has a housing 96 with a substantially conical inner wall 98. One end of the housing 96 is open, at 100, and the other end is closed by a closure plate 102. A screw threaded aperture 104 is provided through the closure plate 102. A resilient collet 106 with central bore 108 sits within the housing 96. A pin 110 with enlarged head seated in a back end of the collet 106 is connected to a thumb screw 112, threadingly engaged in the screw threaded aperture 104. The end of the collet 106 is held to the end of the thumbscrew 112 by the pin 110.

As the thumbscrew 112 is turned to advance it towards the collet 106, the collet is pushed into the reducing diameter of the inner wall and the central bore 108 of the collet reduces for clamping a coiling rod, an example of which is indicated at 114. As the thumbscrew 112 is turned in the other direction, the collet is drawn back into the housing and the central bore 108 enlarges, thereby freeing the coiling rod.

The other end of the coiling rod 114 is non-circular, in this case hexagonal, and locates in the aperture 92 on the underside of the intermediate cap 18. A threaded screw 116 is threaded into an aperture in the end of the housing 96, to one side of the central aperture. The head of the screw 116 acts as a guide for vaping wire 118 being coiled, as described further below. A hole 120 is provided through an upstand in the underside of the intermediate cap 18, which leads from the side of the upstand to a position adjacent the aperture 92 for the coiling rod 114. The hole 120 also serves to guide coiling wire.

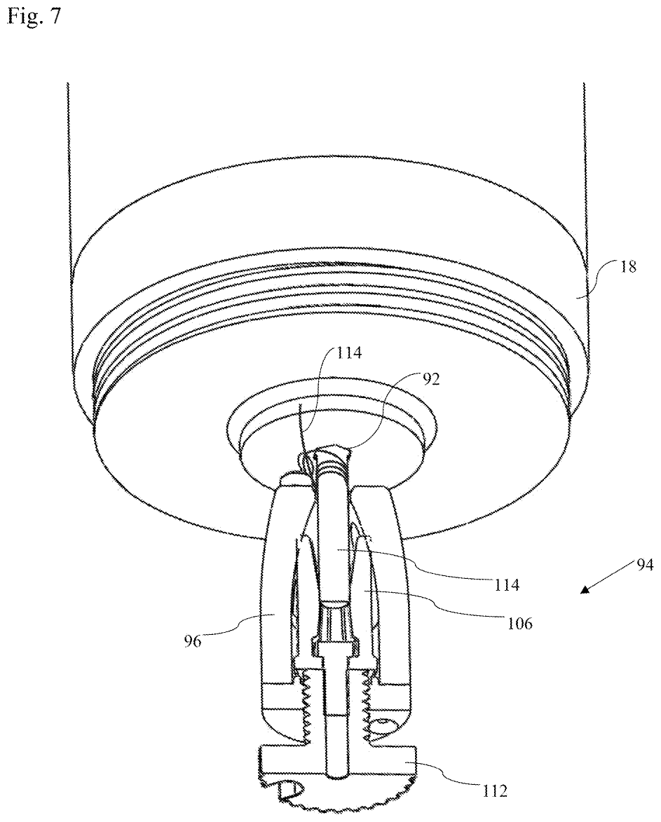

The vape tool kit 10 can be carried easily in a person's pocket. The use of the tool kit 10 will now be described with reference to FIGS. 6 and 7. In use, the upper end cap 16 can be removed from the central body portion 14 and the tool barrel 22 removed. The tool barrel 22 has a flat base and can stand on a table or working surface. The tools can be removed and put away in the barrel as desired.

The intermediate cap 18 is then removed from the central body portion 14 and the spool 44 removed. A portion of wire can be unwound from the spool 44 for use in making coils. The wire can be temporarily wound around the hook shaped protrusions 47 on the outer of the spool 44 to keep it tidy and a short piece cut for making a coil. The spool 44 can then be replaced in the space 76 in the central body portion 14 and the intermediate cap replaced. At this point, the lower end cap 20 is removed to expose the hexagonal aperture 92.

A suitable coiling rod 114 is then selected from the tool barrel and engaged in the hexagonal aperture 92. The piece of wire 118 is fed through the aperture 120 with around 10 mm of wire exposed beneath the aperture. The coiling tool 94 is then removed from the tool barrel 22 and tightened onto the exposed distal end of the coiling rod 114, as previously described using the thumb screw 112. The intermediate end cap 18, central body portion 14 upper end cap 16, and coiling rod 114 are then rotated relative to the coiling tool 94. The head of the screw 116 bears against the wire 118 and the rotation forms a coil of wire around the coiling rod 114. The rotation continues until the desired size of coil is completed. The coiling tool 94 can then be disconnected from the coiling rod 114 and the formed coil removed and trimmed.

A tester, which tests the resistance of the wire by applying a voltage across it, can also usefully be stored in the tool barrel. Before using the coil, its resistance should be tested to make sure that it is safe to use. The completed coil can then be used immediately or stored in the space 85. If the coil is utilised, then it is screwed into a vaping unit in conventional manner, liquid added and cotton fed through the coil. A coil can also be tested in situ, in order to test a coil and vape deck of a vaping device.

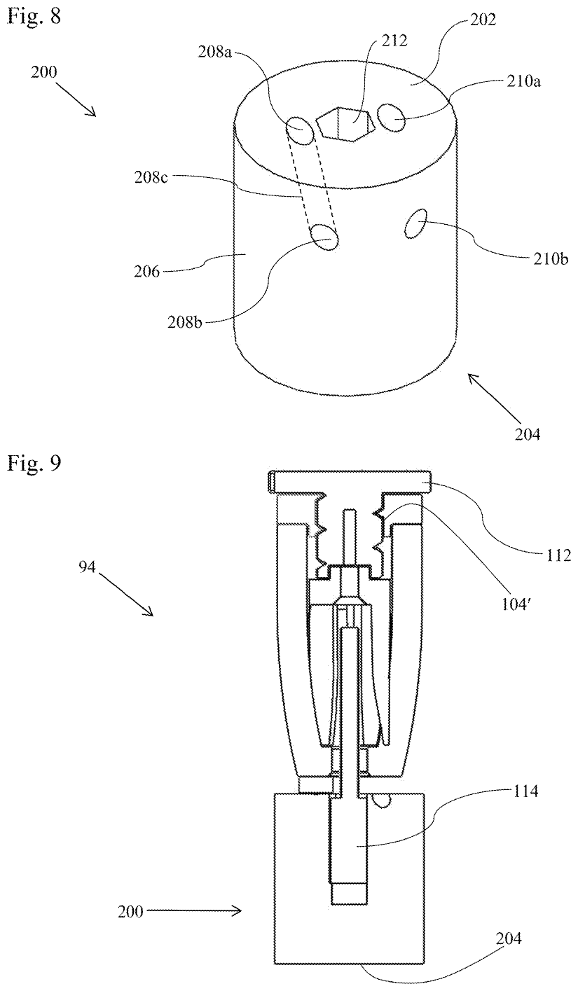

Referring also to FIGS. 8 to 11, components of another embodiment or other embodiments of a vape tool and vape tool kit will now be described.

A coiling rod holder is indicated generally at 200. The coiling rod holder 200 can. be used as a handle for making a coil, that is to say it is handheld. The holder 200 can be controlled or manipulated with one hand whilst the coiling tool 94 can be operated or manipulated with the other hand. The coiling rod holder 200 is more easily held and wielded than the casing with the coiling rod in the intermediate cap and central body portion.

The coiling rod holder 200 is of a similar diameter to the coiling tool 94. The coiling rod holder 200 is about half the length of the coiling tool 94 in this embodiment, but it will be appreciated that the holder 200 may have any suitable length.

The coiling rod holder 200 is substantially cylindrical. The holder 200 includes a first end 202, a second end 204 and a sidewall 206. A coiling rod aperture 212 is provided in the first end. The coiling rod aperture 212 is located substantially centrally in the holder 200. The aperture 212 extends about halfway into the holder 200. The aperture has a hexagonal cross-section in this embodiment.

The holder 200 includes a first pair of holes 208a, 208b for guiding coiling wire like the hole 120 in the earlier embodiment. The holder 200 also includes a second pair of holes 210a, 210b. In the first pair of holes 208a, 208b, one hole 208a is provided in the first end 202 adjacent to the coiling rod aperture 212, and the other hole 208b is provided in the curved sidewall 206. In the second pair of holes 210a, 210b, one hole 210a is provided in the first end 202 adjacent to the coiling rod aperture 212, on the other side of that aperture 212 to the first hole 208a, and the other hole 210b is provided in the curved sidewall 206.

The holes 208a, 208b are linked by a bore (indicated at 208c by dashed lines). The bore 208c extends through the body of the holder 200. In this embodiment, the bore 208c is substantially straight. Similarly, the bore 208c is of substantially constant width in this embodiment. It will be appreciated that the bore may have any form suitable for the coiling wire to pass through. Also, although not shown, a second bore connects the holes 210a, 210b together, and is substantially similar to the bore 208c.

FIG. 9 shows the coiling rod holder 200 in engagement with the coiling rod 114 and coiling tool 94. The process of making a coil using these parts is substantially similar to the embodiment described previously, with the coiling rod holder 200 being used in place of the intermediate cap 18 and other parts.

The coiling tool 94 is broadly similar to the earlier embodiment, but it will be noted that one of the differences is the screw thread (indicated at 104′) for the thumbscrew 112. In this embodiment, there are far fewer turns required to withdraw or replace the thumbscrew because there are a lower number of screw thread turns per unit length along the screw-threaded portion. For example, this may be achieved by having fewer turns or an increased helix angle or increased thread pitch compared to the earlier embodiment.

In FIG. 9, the screw thread 104′ spirals around the thumbscrew 112. In this case, the crests of the thread 104′ are relatively elongate, and longer than the thread is deep (or greater than the distance between adjacent roots on the same side of the thread 104′). About 2 to 3 turns of the thumbscrew 112 would be enough to fully withdraw or insert the thumbscrew 112.

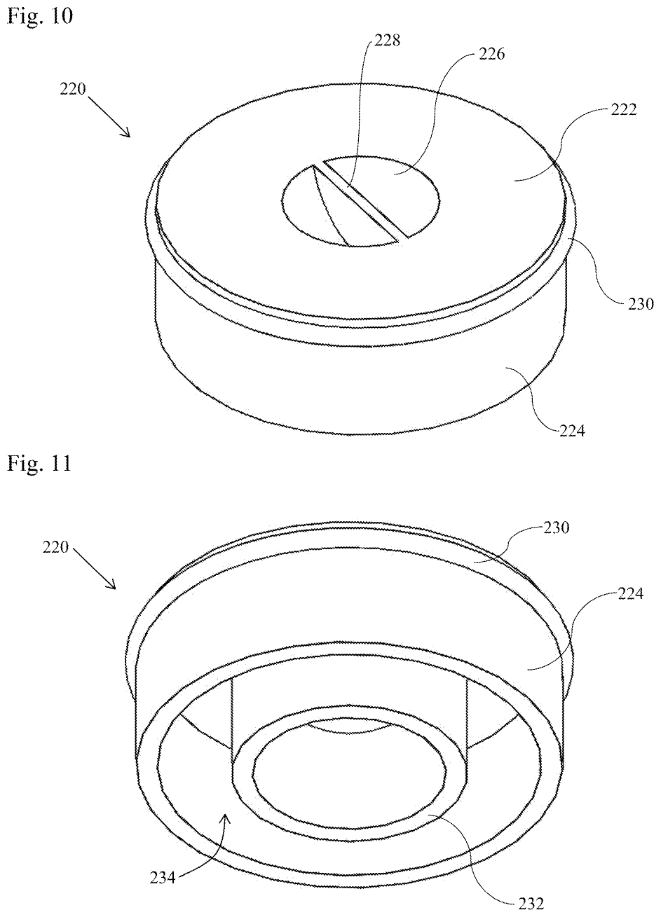

Referring also to FIGS. 10 and 11, a cover is indicated generally at 220. The cover 220 may be considered as a storage cap or intermediate cap which can be placed in another version of the lower end cap (not shown). Instead of providing internal structure in the lower end cap 20 and a cover plate 86, the cover 220 itself can be shaped to provide the necessary storage space for cotton, for example. This simplifies the structure of the lower end cap.

The cover 220 includes a top surface 222 and a sidewall 224 connected to the top surface 222. The sidewall 224 is curved in this embodiment. The cover 220 is approximately cylindrical for fitting within the lower end cap. A circumferential seal 230 is provided around the upper end of the sidewall 224, next to the top surface 222.

A recess 226 is provided in the top surface 222. A handle member 228 is provided across the recess 226 to allow the cover 220 to be withdrawn from or placed into the lower end cap. It will be appreciated that a spigot is not required in this instance. The intermediate cap may also be omitted in some embodiments, because there is no spigot to accommodate when assembling the parts for storage.

The handle member 228 is in the form of a divider or wall. The depth of the recess 226 and size of the wall 228 is sufficient for the wall to be grasped by a thumb and finger, or by two fingers.

A secondary wall 232 is provided within and spaced from the sidewall 224. A chamber or storage space is indicated generally at 234. The chamber 234 is defined by the inner surface of the sidewall 224, the outer surface of the secondary wall 232, and the underside of the top surface 222. Pre-formed coils and other parts may be stored in the space within the secondary wall 232 in a similar manner to the earlier embodiment.

The embodiments described above are provided by way of example only, and various changes and modifications will be apparent to persons skilled in the art without departing from the scope of the present invention as defined by the appended claims.

1. A vape tool comprising a coiling rod, and a coiling tool for engaging with and rotating around the coiling rod,
the coiling tool having a housing with a substantially conical inner wall, a collet disposed inside the housing and arranged to bear against the inner wall, an internal diameter of the collet being determined by a longitudinal position of the collet within the housing and a control means for controlling the longitudinal position of the collet within the housing.

2. The vape tool as claimed in claim 1, in which the control means is a thumb screw threadingly engaged with the housing and connected to the collet.

3. The vape tool as claimed in claim 1, in which an aperture is provided in an end of the housing for receiving the coiling rod.

4. The vape tool as claimed in claim 1, in which a coiling rod holder is provided for use with the coiling rod and coiling tool.

5. The vape tool as claimed in claim 4, in which the coiling rod holder is substantially cylindrical.

6. The vape tool as claimed in claim 4, in which a coiling rod aperture is provided in an end of the coiling rod holder for receiving the coiling rod.

7. The vape tool as claimed in claim 6, in which a first hole for guiding coiling wire is provided adjacent to the coiling rod aperture, and a first corresponding hole is provided on a first side or another end of the coiling rod holder and connected to the hole through the body of the coiling rod holder.

8. The vape tool as claimed in claim 7, in which a second hole is provided adjacent to the coiling rod aperture, opposite the first hole, and a second corresponding hole is provided on a second side or another end of the coiling rod holder and connected to the second hole through the body of the coiling rod holder.

9. The vape tool as claimed in claim 6, in which the coiling rod aperture includes a substantially hexagonal or non-circular portion for receiving a substantially hexagonal or non-circular end of the coiling rod.

10. The vape tool as claimed in claim 1, in which a wire guide is disposed at an end of the housing, for guiding wire around the coiling rod on rotation of the coiling tool relative to the coiling rod.

11. The vape tool as claimed in claim 10, in which the wire guide is a screw.

12. (canceled)

13. (canceled)

14. (canceled)

15. (canceled)

16. (canceled)

17. (canceled)

18. (canceled)

19. (canceled)

20. (canceled)

21. (canceled)

22. A vape tool kit comprising a casing including a central body portion and an upper end cap attachable to one end of the central body portion and enclosing a first space, an intermediate cap enclosing a second space, and a lower end cap enclosing a third space, a tool kit being located in the first space, a spool and sealable cotton storage area being located in the second and third spaces, in which the intermediate cap is attachable to the other end of the central body portion and the lower end cap is attachable to the intermediate cap, or in which the intermediate cap is positionable in the lower end cap and the lower end cap is attachable to the central body portion.

23. The vape tool kit as claimed in claim 22, in which the tool kit includes a cylindrical barrel, having apertures formed therein for receiving tools.

24. (canceled)

25. The vape tool kit as claimed in claim 22, in which the cylindrical barrel is disposed within the first space.

26. The vape tool kit as claimed in claim 22, in which the spool is formed from upper and lower parts and includes a U-shaped circular wall onto which wire can be coiled.

27. The vape tool kit as claimed in claim 26, in which the spool has a cover element which lies over the U-shaped circular wall for protecting coiled wire on the spool.

28. The vape kit as claimed in claim 26, in which external protrusions are provided on the spool for temporarily receiving a second coil of wire.

29. The vape kit as claimed in claim 22, in which a cover or cover plate closes a space in the lower end cap and forms the sealable cotton storage area, in which the cover or cover plate is provided with a peripheral seal.

30. (canceled)

31. The vape kit as claimed in claim 22, in which the upper end cap is screwed to one end of the central body portion, and either the intermediate cap is screwed to the other end of the central body portion and the lower end cap is screwed to the intermediate cap, or the intermediate cap is disposable inside the lower end cap and the lower end cap is screwed to the other end of the central body portion.

32. A vape tool kit comprising a casing including a central body portion and an upper end cap attachable to one end of the central body portion and enclosing a first space, an intermediate cap attachable to the other end of the central body portion and enclosing a second space, and a lower end cap attachable to the intermediate cap and enclosing a third space, a tool kit being located in the first space, a spool and sealable cotton storage area being located in the second and third spaces.
